(19) United States
(12) Patent Application Publication
(10) Pub. No.: US 2009/0030052 A1
(43) Pub. Date: Jan. 29, 2009

(54) PHARMACEUTICAL TABLET COMPOSITIONS CONTAINING IRBESARTAN

(75) Inventors: Rajesh Kshirsagar, Vadodara (IN); Sachin Mundade, Vadodara (IN); Ranadheer Reddy, Vadodara (IN)

Correspondence Address: VOLPE AND KOENIG, P.C., UNITED PLAZA, SUITE 1600, 30 SOUTH 17TH STREET, PHILADELPHIA, PA, 19103, US

(73) Assignee: ALEMBIC LIMITED, Gujarat (IN)

(21) Appl. No.: 12/279,683

(22) PCT Filed: Feb. 19, 2007
(86) PCT No.: PCT/IN07/00072

§ 371 Date: Aug. 15, 2008

(30) Foreign Application Priority Data
Feb. 17, 2006 (IN) 226/MUM/2006

(51) Int. Cl. A61K 31/41; A61P 9/12

(52) U.S. Cl. 514/381

(57) ABSTRACT

A pharmaceutical tablet composition comprising irbesartan and lactose, said composition being essentially free of surfactant.

### FIELD OF INVENTION:

The present invention relates to pharmaceutical tablet compositions comprising irbesartan and lactose and essentially free of surfactant.

### BACKGROUND OF THE INVENTION

Irbesartan is chemically known as 2-butyl-3-[[29-(1H-tetrazol-5-yl) [1,19-biphenyl]-4-yl]methyl]1,3-diazaspiro[4,4] non-1-en-4-one, also as 2-n-butyl-4-spirocyclopentane-1-[(2′-(tetrazol-5- yl)biphenyl-4-yl)methyl]-2-imidazolin-5-one, also as 2-butyl-3-[[2′-(1 H-tetrazol-5-yl) [1,1′-biphenyl]-4-y]methyl]-1,3-diazaspiro [4,4]non-1-en-4-one. Its empirical formula is C₂₅H₂₈N₆₀, and it has the structure given in Formula 1.

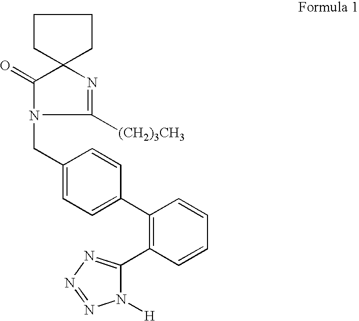

Irbesartan has a molecular weight of 428.5. The compound is described in U.S. Pat. No. 5,270,317.

In the United States, irbesartan is available for oral administration tablets containing 75 mg, 150 mg, or 300 mg of irbesartan, which are sold under the brand name AVAPRO®.

U.S. Pat. No. 6,342,247, describes irbesartan as a fluffy material, with relatively low bulk and tap densities. The patent further states that these properties make it difficult to formulate a large amount of the drug into a small tablet with uniformity of weight, hardness, and other desirable tablet properties. In addition, irbesartan has certain undesirable flow characteristics, for example, is sticky and can adhere to surfaces such as tablet punch faces and dies, causing problems in tableting, especially on a high speed tablet press. The low aqueous solubility of irbesartan also presents a challenge, since, to keep the tablet mass small, only limited amounts of excipients may be added to facilitate wetting, disintegration, and ultimately, rapid and complete drug release. The '247 patent claims an oral formulation of irbesartan containing a surfactant i. e. Poloxamer 188 as an essential component of the formulation. According to '247 the Poloxamer surfactant improves the aqueous granulation of irbesartan (which is hydrophobic), eases the ejection of tablets after compression and accelerates the dissolution of irbesartan active agent.

The present inventors have now surprisingly found that pharmaceutical composition comprising irbesartan and lactose can be prepared without incorporation of surfactant yet have good disintegration and dissolution properties.

### SUMMARY OF THE INVENTION

The present invention provides pharmaceutical tablet compositions comprising irbesartan and lactose and essentially free of surfactant yet have excellent properties for tablet formation, and which give rapid and complete drug release.

In one embodiment, the present invention provides a pharmaceutical tablet composition comprising irbesartan and lactose and essentially free of surfactant

In yet another embodiment, the present invention provides a pharmaceutical tablet composition comprising irbesartan and lactose and essentially free of surfactant and which is prepared by wet granulation.

In yet another embodiment, the present invention provides a pharmaceutical tablet composition comprising irbesartan and lactose and essentially free of surfactant and which is prepared by dry granulation.

In yet another embodiment, the invention provides a pharmaceutical tablet composition comprising irbesartan and lactose and essentially free of surfactant and which exhibits a dissolution profile such that greater than about 70% of the irbesartan is dissolved within about 30 minutes.

### DETAILED DESCRIPTION OF THE INVENTION

This invention relates to pharmaceutical tablet compositions comprising irbesartan and lactose and essentially free of surfactant. It is surprising that even without surfactant, these compositions have excellent properties for tablet formation, and which gives rapid and complete drug release. Whilst not wishing to be bound by theory it is felt that lactose along with irbesartan in a ratio (when present in a particular ratio of irbesartan to lactose ranging from 4:1 to 1:4) yield microgranules when granulated. These microgranules are capable of improving flow and ejection of tablets of irbesartan which is inherently a sticky material but being small do not retard dissolution.

Unless otherwise indicated, mention of irbesartan herein also includes pharmaceutically acceptable salts thereof.

The pharmaceutical tablet compositions comprising irbesartan and lactose and essentially free of surfactant wherein the ratio of irbesartan to lactose is ranging from 4:1 to 1:4. The lactose of the invention is preferably lactose monohydrate. In a preferred embodiment the lactose is a mixture of two grades of lactose monohydrate i. e. Granulac® and Flowlac®.

In a preferred embodiment, the invention provides pharmaceutical tablet compositions comprising irbesartan and lactose and essentially free of surfactant wherein the irbesartan is present at concentrations less than 80% w/w composition, more preferably the composition comprise from about 20% w/w to about 60% w/w irbesartan.

In addition to irbesartan and lactose the compositions may comprise one or more pharmaceutically acceptable excipients. In this context, the term “excipient” refers to pharmaceutically acceptable materials known to those of ordinary skilled in the art of pharmacy to aid the administration of the medicinal agent. Excipients for inclusion in the compositions of the invention include but are not limited to binders, disintegrants, antiadherants, lubricants, coloring agents and the like.

Examples of binders include but are not limited to alginic acid or sodium alginate, cellulose or cellulose derivatives such as carboxymethylcellulose sodium, ethylcellulose, hydroxyethyl cellulose, hydroxypropyl cellulose, hydroxypropyl methylcellulose, or methylcellulose, gelatin, povidone (polyvinylpyrrolidone i.e., 1-ethenyl-2-pyrrolidinone homopolymer), starch, pregelatinized starch and the like.

Examples of disintegrants include but are not limited to alginic acid or sodium alginate, cellulose or cellulose derivatives such as carboxymethylcellulose sodium, microcrystalline cellulose, powdered cellulose, croscarmellose sodium (cross-linked polymer of carboxymethylcellulose sodium), crospovidone (cross-linked homopolymer of N-vinyl-2-pyrrolidinone i.e. cross-linked 1-ethenyl-2-pyrrolidinone), pregelatinized starch, sodium starch glycolate, starch and the like.

Examples of antiadherants include but not limited to silicon-containing compounds such as silicon dioxide, magnesium trisilicate, talc and the like.

Examples of lubricants include but are not limited to fatty acids or fatty acid derivatives such as calcium stearate, glyceryl monostearate, glyceryl palmitostearate, magnesium stearate, sodium lauryl sulfate, sodium stearyl fumarate, zinc stearate or stearic acid, hydrogenated vegetable oil, polyalkylene glycols such as polyethylene glycol, sodium benzoate, talc and the like.

Examples of coloring agents include but are not limited to ferric oxides and the like.

As can be seen from the above, a single compound may perform two or more functions. All these excipients are used in ranges well known to the person skilled in the art. Calculation of weight percent is preferably on the basis of the primary function of a compound in a given composition.

In a preferred embodiment a pharmaceutical tablet composition comprising irbesartan and lactose and essentially free of surfactant also comprises of a disintegrant and a lubricant.

In a yet preferred embodiment, a pharmaceutical tablet composition comprising irbesartan and lactose and essentially free of surfactant comprises a disintegrant such as croscarmellose sodium or sodium starch glycolate, and a lubricant such as magnesium stearate.

In a still preferred embodiment, a pharmaceutical tablet composition comprising irbesartan and lactose and essentially free of surfactant comprises of about 40% to 60% w/w of the composition of irbesartan, about 30% to 50% w/w of the composition of lactose monohydrate, about 1% to 10% w/w of the composition of croscarmellose sodium and about 0.5 to 5% w/w of the composition of magnesium stearate.

The pharmaceutical tablet composition comprising irbesartan and lactose and essentially free of surfactant can be prepared by a suitable granulation method well known in the art. For example tablets can be prepared by wet granulation, dry granulation Wet granulation may be carried out, using aqueous and/or non aqueous solvents. Examples of solvents used as granulating fluids include but are not limited to methylene chloride, isopropyl alcohol, acetone, methanol, ethanol, water or mixtures thereof. Dry granulation may be carried out, for example, by using a roller compactor or alternatively, for example, by the process of slugging.

In a preferred embodiment a pharmaceutical tablet composition comprising irbesartan and lactose and essentially free of surfactant is prepared by a wet granulation process as follows: Irbesartan, lactose monohydrate (Granulac® and Flowlac®) and a portion of the croscarmellose sodium are sized and mixed. Above powder blend is granulated by adding sufficient quantity of water or povidone dissolved in water. The granules obtained are dried until the loss on drying (LOD) is 2% or less. The dried granules are mixed with croscarmellose sodium. The blend obtained is then mixed with magnesium stearate, lubricated and compressed into tablets.

In yet another preferred embodiment a pharmaceutical tablet composition comprising irbesartan and lactose and essentially free of surfactant is prepared by using dry granulation. Irbesartan, lactose monohydrate (Granulac® and Flowlac®) and a portion of the sodium starch glycolate are sized and mixed. Above powder blend is compacted into slugs. The slugs obtained are milled, sized and mixed with sodium starch glycolate. The blend obtained is then mixed with the magnesium stearate and lubricated. The lubricated blend is compressed into tablets.

In another embodiment the pharmaceutical tablet composition comprising irbesartan and lactose and essentially free of surfactant exhibits a dissolution profile such that about 70% of irbesartan is dissolved within 30 minutes when these tablets are tested using USP apparatus 2, in 1000 mL of 0.1N hydrochloric acid at 37° C. with paddle speed of 50 rpm.

In a preferred embodiment of the invention, the pharmaceutical tablet composition comprising irbesartan and lactose and essentially free of surfactant exhibits a dissolution profile such that greater than about 85% of the irbesartan is dissolved within about 30 minutes, when these tablets are tested using USP apparatus 2, in 1000 mL of 0.1N hydrochloric acid at 37° C. with paddle speed of 50 rpm.

The present pharmaceutical tablet compositions comprising irbesartan and lactose and essentially free of surfactant can be reproducibly manufactured on a large scale.

The present pharmaceutical tablet compositions comprising irbesartan and lactose and essentially free of surfactant for example, can be compressed on high speed tableting equipment (especially, a high speed tablet press) to form tablets which are uniform in both weight and content and which exhibit desirable physical properties, including elegant appearance, low friability, and fast disintegration time. Tablets prepared from the present compositions are capable of releasing the active component(s), by dissolution, in a fast and reproducible manner.

The following examples are provided to illustrate preferred embodiments of the invention, and are not intended to limit the scope of the present invention.

### EXAMPLE 1

### Preparation of Irbesartan Tablets by Wet Granulation

TABLE 1

| | | |
|---|---|---|
| Ingredients | Percent (%) w/w | Category/Role |

Intragranular

| | | |
|---|---|---|
| Irbesartan | 50.00 | Active Ingredient |
| Lactose Monohydrate | 22.33 | Diluent |
| (Granulac ®) | | |
| Lactose Monohydrate | 22.25 | Diluent |
| (Flowlac ®) | | |
| Croscarmellose sodium | 2.66 | Disintegrant |
| Water | q.s. | Granulating fluid |

Extragranular

| | | |
|---|---|---|
| Croscarmellose sodium | 2.00 | Disintegrant |
| Magnesium stearate | 0.75 | Lubricant |
| Total | 100 | — |

Using the above composition, the tablets were prepared by a wet granulation process as follows. Irbesartan, lactose monohydrate (Granulac® and Flowlac®) and a portion of the croscannellose sodium were sized and mixed. Above powder blend was granulated by adding sufficient quantity of water. The granules obtained were dried until the loss on drying (LOD) was 2% or less. The dried granules were mixed with croscarmellose sodium. The blend obtained was then mixed with magnesium stearate, lubricated and compressed into tablets.

### EXAMPLE 2

### Preparation of Irbesartan Tablets by Dry Granulation

TABLE 2

| | | |
|---|---|---|
| Ingredients | Percent (%) w/w | Category/Role |

Intragranular

| | | |
|---|---|---|
| Irbesartan | 50.00 | Active Ingredient |
| Lactose Monohydrate | 22.75 | Diluent |
| (Granulac ®) | | |
| Lactose Monohydrate | 23.50 | Diluent |
| (Flowlac ®) | | |
| Sodium Starch Glycolate | 2.66 | Disintegrant |
| (Glycolys ®) | | |

Extragranular

| | | |
|---|---|---|
| Sodium Starch Glycolate | 1.33 | Disintegrant |
| (Glycolys ®) | | |
| Magnesium stearate | 0.75 | Lubricant |
| Total | 100 | — |

The tablets were prepared using dry granulation. Irbesartan, lactose monohydrate (Granulac® and Flowlac®) and a portion of the sodium starch glycolate were sized and mixed. Above powder blend was compacted into slugs. The slugs obtained were milled and mixed with sodium starch glycolate. The blend obtained was then mixed with the magnesium stearate and lubricated. The lubricated blend was compressed into tablets.

### EXAMPLE 3

### Preparation of Irbesartan Tablets by Wet Granulation

The composition of table 2 was used to prepare tablets by wet granulation process described in Example 1. Water was used as the granulating fluid.

### EXAMPLE 4

### Preparation of Irbesartan Tablets by Wet Granulation

TABLE 3

| | Category/ | Percent (%)w/w | | | | | |
|---|---|---|---|---|---|---|---|
| Ingredients | Role | A | B | C | D | E | F |

Intragranular

| | | | | | | | |
|---|---|---|---|---|---|---|---|
| Irbesartan | Active | 77.00 | 40.00 | 60.00 | 40.00 | 60.00 | 76.92 |
| | Ingredient | | | | | | |
| Lactose | Diluent | 18.40 | 26.60 | 18.0 | 26.60 | 18.00 | 16.15 |
| Monohydrate | | | | | | | |
| (Granulac ®) | | | | | | | |
| Lactose | Diluent | — | 27.80 | 16.60 | 27.80 | 16.60 | — |
| Monohydrate | | | | | | | |
| (Flowlac ®) | | | | | | | |
| Croscarmellose | Disintegrant | 2.50 | 2.66 | 2.80 | — | — | 2.56 |
| sodium | | | | | | | |
| Sodium starch | Disintegrant | — | — | — | 2.66 | 2.80 | — |
| glycolate | | | | | | | |
| (Glycolis ®) | | | | | | | |
| Polyvinylpyrrolidone | Binder | — | — | — | — | — | 2.31 |
| K-30 | | | | | | | |
| Water | Granulating | q.s. | q.s. | q.s. | q.s. | q.s. | q.s. |
| | fluid | | | | | | |

Extragranular

| | | | | | | | |
|---|---|---|---|---|---|---|---|
| Croscarmellose | Disintegrant | 1.30 | 2.00 | 1.80 | — | — | 1.29 |
| sodium | | | | | | | |
| Sodium starch | Disintegrant | — | — | — | 2.00 | 1.80 | — |
| glycolate | | | | | | | |
| (Glycolis ®) | | | | | | | |
| Magnesium stearate | Lubricant | 0.75 | 0.80 | 0.80 | 0.80 | 0.80 | 0.77 |
| Total | — | 100 | 100 | 100 | 100 | 100 | 100 |

Using the above compositions, the tablets were prepared by a wet granulation process as follows. Irbesartan, lactose monohydrate (Granulac® and/or Flowlac® and a portion of the croscamellose sodium or sodium starch glycolate were sized and mixed. Above powder blend was granulated by adding sufficient quantity of water or PVP K-30 dissolved in water. The granules obtained were dried until the loss on drying (LOD) was 2% or less. The dried granules were mixed with croscarmellose sodium or sodium starch glycolate. The blend obtained was then mixed with the magnesium stearate, lubricated and compressed into tablets.

Tablets of Example 1 to Example 4 were tested for dissolution according to the U.S. Pharmacopoeia, using USP apparatus 2 with 1000 ml of 0.1 N hydrochloric acid at 37° C. with paddle speed of 50 rpm. The comparative dissolution results of present invention with the AVAPRO® tablets which contain surfactant (Table 4) are set forth in Table 5.

TABLE 4
Qualitative composition of AVAPRO ® (Bristol-Myers
Squibb Sanofi-Synthelabo)

| Ingredients | Category/Role |
|---|---|
| Irbesartan | Active Ingredient |
| Lactose | Diluent |
| Microcrystalline cellulose | Diluent |
| Poloxamer 188 | Surfactant |
| Pregelatinized starch | Binder |
| Colloidal anhydrous silica | Antiadherant |
| Croscarmellose sodium | Disintegrant |
| Magnesium stearate | Lubricant |

TABLE 5
% Cumulative Drug release

| Time | | | | Ex 4 | | | | | | Ex 5 |
|---|---|---|---|---|---|---|---|---|---|---|
| Interval | Ex 1 | Ex 2 | Ex 3 | A | B | C | D | E | F | AVAPRO ® |
| 5 Min | 70.9 | 52.5 | 54.6 | 52.0 | 63.3 | 55.0 | 45.0 | 33.0 | 48.92 | 63.0 |
| 10 Min | 89.0 | 75.3 | 78.7 | 75.0 | 81.0 | 76.0 | 67.0 | 57.0 | 77.30 | 86.0 |
| 15 Min | 94.4 | 86.3 | 89.9 | 85.0 | 88.0 | 85.0 | 79.0 | 72.0 | 87.87 | 93.0 |
| 20 Min | 96.4 | 92.8 | 95.4 | 90.0 | 92.0 | 90.0 | 86.0 | 81.0 | 92.90 | 95.0 |
| 30 Min | 97.7 | 97.0 | 99.6 | 93.0 | 95.0 | 94.0 | 93.0 | 92.0 | 97.60 | 98.0 |
| 45 Min | 98.2 | 99.4 | 101.1 | 96.0 | 98.0 | 95.0 | 97.0 | 97.0 | 98.68 | 99.0 |
| 60 Min | 98.3 | 100.2 | 101.4 | 96.0 | 99.0 | 96.0 | 99.0 | 99.0 | 98.84 | 100.0 |

### EX—EXAMPLE

Thus it is evident from the results in above Table 5 that the compositions of present invention shows comparable release profile with that of Avapro®. Thus even though the composition of present invention lacks the surfactant, which in Avapro® accelerates the dissolution of irbesartan active agent, it achieves similar release profile

1. A pharmaceutical tablet composition comprising irbesartan and lactose said composition being essentially free of surfactant.

2. A pharmaceutical tablet composition comprising irbesartan according to claim 1 which exhibits dissolution profile such that greater than about 70% of the irbesartan is dissolved within about 30 minutes when the tablet composition is tested using USP apparatus 2, placing the tablet in 1000 mL of 0.1N hydrochloric acid at 37° C. with paddle speed of 50 rpm.

3. A pharmaceutical tablet composition comprising irbesartan according to claim 1 wherein irbesartan is present less than 80% w/w of the composition.

4. A pharmaceutical tablet composition comprising irbesartan according to claim 1 wherein irbesartan is present from about 20% w/w to about 60% w/w of the composition.

5. A pharmaceutical tablet composition comprising irbesartan according to claim 1 wherein tablet is prepared by wet granulation.

6. A pharmaceutical tablet composition comprising irbesartan according to claim 1 wherein tablet is prepared by dry granulation.

7. A pharmaceutical tablet composition comprising irbesartan according to claim 1 wherein the ratio of irbesartan to lactose is ranging from 4:1 to 1:4.

8. A pharmaceutical tablet composition comprising,
a) irbesartan
b) lactose
c) disintegrant
d) lubricant and optionally
e) binder and
f) coloring agent
wherein, said composition is essentially free of surfactant.

9. A pharmaceutical tablet composition comprising irbesartan according to claim 8 wherein irbesartan is present less than 80% w/w of the composition.

10. A pharmaceutical tablet composition comprising irbesartan according to claim 8 wherein irbesartan is present from about 20% w/w to about 60% w/w of the composition.

11. A pharmaceutical tablet composition comprising irbesartan according to claim 8 wherein tablet is prepared by wet granulation.

12. A pharmaceutical tablet composition comprising irbesartan according to claim 8 wherein tablet is prepared by dry granulation.

13. A pharmaceutical tablet composition comprising irbesartan according to claim 8 wherein the lactose is lactose monohydrate.

14. A pharmaceutical tablet composition comprising irbesartan according to claim 8 wherein the disintegrant is croscarmellose sodium.

15. A pharmaceutical tablet composition comprising irbesartan according to claim 8 wherein disintegrant is sodium starch glycolate.

16. A pharmaceutical tablet composition comprising irbesartan according to claim 8 wherein the binder is polyvinyl pyrrolidone.

17. A pharmaceutical tablet composition comprising irbesartan according to claim 8 wherein the lubricant is magnesium stearate.

18. A pharmaceutical tablet composition comprising irbesartan according to claim 8 wherein the ratio of irbesartan to lactose is ranging from 4:1 to 1:4.
